(19) United States
(12) Patent Application Publication
(10) Pub. No.: US 2020/0155345 A1
(43) Pub. Date: May 21, 2020

(54) COLD LEG WRAP

(71) Applicant: Phil Gaimon, LLC, N Hollywood, CA (US)

(72) Inventor: Phil Gaimon, N Hollywood, CA (US)

(21) Appl. No.: 16/688,926

(22) Filed: Nov. 19, 2019

Related U.S. Application Data
(60) Provisional application No. 62/769,988, filed on Nov. 20, 2018.
(60) Provisional application No. 62/869,150, filed on Jul. 1, 2019.

(51) Int. Cl. A61F 7/10; A61F 7/02

(52) U.S. Cl. CPC A61F 7/10; A61F 13/622; A61F 7/02

(57) ABSTRACT

A leg wrap has an elongate body portion having an outer layer and an inner layer that define one or more interior spaces configured to receive one or more cold packs. Preferably, the body portion is sized to cover at least a substantial entirety of a length of the user's leg and, when wrapped around the user's leg, to cover at least a substantial entirety of a circumference of the user's leg. In some configurations, multiple sizes of the leg wrap can be provided to suit a range of users. The leg wrap also includes a plurality of retention elements configured to retain the leg wrap in the wrapped orientation on the user's leg.

### BACKGROUND

### Field

The present disclosure relates to cold application devices for athletic recovery or other therapeutic purposes. In particular, the present disclosure relates to a wrap device that holds cold elements (e.g., ice bags or ice packs) against a user's limb (e.g., leg).

### Description of the Related Art

Cold therapy is often used for athletic recovery and to reduce pain, swelling and nerve activity. Methods of cold therapy include ice baths and cyro chambers, which are inconvenient and can be expensive, especially if ice needs to be purchased to obtain a suitable quantity.

### SUMMARY

Therefore, a need exists for more convenient and/or less expensive options for cold therapy. The systems, methods and devices described herein have innovative aspects, no single one of which is indispensable or solely responsible for their desirable attributes. Without limiting the scope of the claims, some of the advantageous features will now be summarized.

A leg wrap has an elongate body portion having an outer layer and an inner layer that define one or more interior spaces configured to receive one or more cold packs. Preferably, the body portion is sized to cover at least a substantial entirety of a length of the user's leg and, when wrapped around the user's leg, to cover at least a substantial entirety of a circumference of the user's leg. In some configurations, multiple sizes of the leg wrap can be provided to suit a range of users. The leg wrap also includes a plurality of retention elements configured to retain the leg wrap in the wrapped orientation on the user's leg.

In some implementations, the interior space of the body portion is divided into a plurality of discrete spaces or chambers, each configured to receive a cold pack. The leg wrap can include a reinforcement strip that extends in a lengthwise direction along the body portion. The attachment of the reinforcement strip can couple the outer layer and inner layer to segment the interior space into two sides. The leg wrap can also include a plurality of segment lines (e.g., stitch lines) that couple the outer layer and the inner layer to create multiple chambers or pockets on one or both sides created by the reinforcement strip. In some implementations, two or three stitched segment lines are provided to create three or four chambers on each side for a total of six or eight chambers. However, other suitable number of chambers can be provided using any desired number of reinforcement strips (or other longitudinal segment lines) and lateral segment lines.

The leg wrap includes one or more openings that permit access to the interior chambers. In some implementations, a longitudinal access opening is provided on each side of the body portion. Each access opening permits access to all of the chambers on the side of the access opening. The access openings can be defined by or within either or both of the outer layer and the inner layer. In some implementations, the access openings are defined by or within the outer layer. The access openings can be selectively opened and closed. In some implementations, each of the access openings includes a zipper that allows for access and closure.

In some implementations, the retention elements are bands or straps. The bands or straps can be elastic straps. Each of the bands or straps can have a first portion on a first side of the body portion and a second portion on a second side of the body portion. The first portion and the second portion can be coupled to one another. In some implementations, a hook-and-loop fastener is used to couple the first portion and the second portion of the band or strap.

In some implementations, the retention elements are bands or straps. Each of the bands or straps can extend along one side of the body portion and connect to the other side of the body portion. An attached end of each of the bands or straps can be attached at or near a center of the body portion and a remainder of each of the bands or straps is free of the body portion. A length of each of the bands or straps can be greater than one-half of a width of the body portion such that each of the bands or straps overhangs the body portion when the leg wrap is laid flat. Each of the bands or straps can include one or more fastener strips oriented in a lengthwise direction of the band or strap, the fastener strips configured to attach to the body portion. The body portion can include one or more fastener strips oriented in a lengthwise direction of the body portion such that the fastener strips of the bands or straps are perpendicular or substantially perpendicular to the fastener strips of the body portion when the bands or straps are attached to the body portion. The fastener strips of the bands or straps can be hook material and the fastener strips of the body portion can be loop material of a hook and loop fastener. In some implementations, one of the bands or straps is provided for each pocket in a lengthwise direction of the body portion.

In some implementations, a pair of leg wraps are sold as a kit to permit the user to apply cold therapy to both legs at the same time. Cold packs can be provided with the leg wrap and configured such that each one of the cold packs is received within one of the chambers or pockets. The wrap could also be configured for use on the user's arm or other body portion (e.g., torso).

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through the use of the accompanying drawings.

FIG. 1 is a view of an outer layer of a leg wrap in an unwrapped, laid flat configuration.

FIG. 2 is a view of an inner layer of the leg wrap of FIG. 1.

FIG. 3 is a view of an outer layer of an alternative embodiment of a leg wrap in an unwrapped, laid flat configuration.

FIG. 4 is a view of the outer layer of the leg wrap of FIG. 3 with retention straps folded over to show an inner surface of the straps.

FIG. 5 is a view of an inner layer of the leg wrap of FIG. 3.

FIG. 6 is a view of the leg wrap of FIG. 3 in a wrapped configuration.

FIG. 7 is a view of the outer layer of the leg wrap of FIG. 3 with ice packs or cold packs removed from the leg wrap.

FIG. 8 is an enlarged view of a portion of the leg wrap and ice packs or cold packs of FIG. 7.

### DETAILED DESCRIPTION

Embodiments of systems, components and methods of assembly and manufacture will now be described with reference to the accompanying figures, wherein like numerals refer to like or similar elements throughout. Although several embodiments, examples and illustrations are disclosed below, it will be understood by those of ordinary skill in the art that the inventions described herein extends beyond the specifically disclosed embodiments, examples and illustrations, and can include other uses of the inventions and obvious modifications and equivalents thereof. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

Certain terminology may be used in the following description for the purpose of reference only, and thus are not intended to be limiting. For example, terms such as “above” and “below” refer to directions in the drawings to which reference is made. Terms such as “front,” “back,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

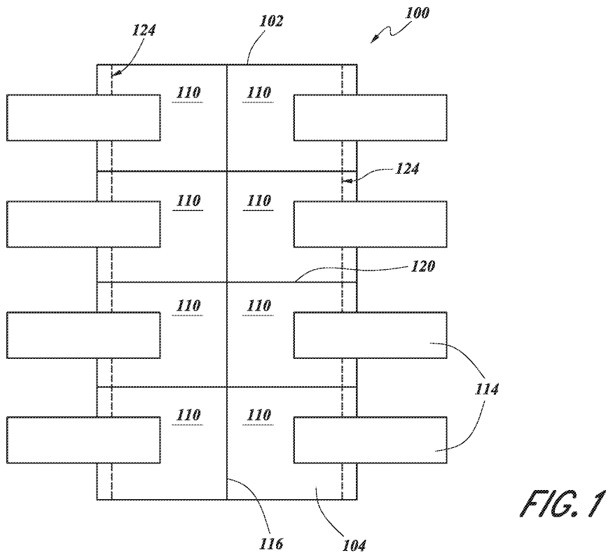

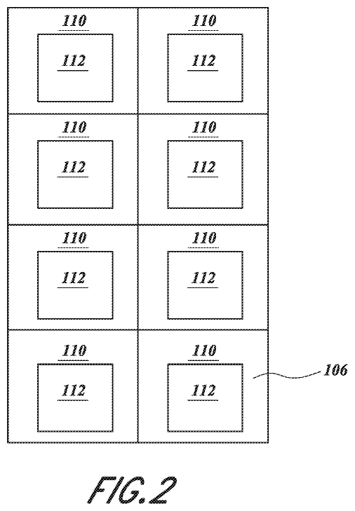

FIGS. 1 and 2 illustrate a leg wrap 100, which has an elongate body portion 102 having an outer layer 104 (FIG. 1) and an inner layer 106 (FIG. 2) that define one or more interior spaces, chambers or pockets 110 configured to receive one or more cold packs 112. The outer layer 104 can be thicker or less flexible than the inner layer 106. In some implementations, the outer layer 104 is a thick canvas, similar or other suitable material (e.g., 400 denier CORDURA™ nylon) and the inner layer is a thin fabric (e.g., nylon pack cloth) material. Such an arrangement allows the outer layer 104 to advantageously hold the cold packs 112 against the user's leg and the inner layer 106 to be comfortable and conformable against the user's leg.

Preferably, the body portion 102 is sized to cover at least a substantial entirety of a length of the user's leg and, when wrapped around the user's leg, to cover at least a substantial entirety of a circumference of the user's leg. In some cases, the body portion 102 can overlap itself when wrapped around the user's leg. For example, the body portion 102 can have a lengthwise dimension of about 20-30 inches or about 24-27 inches. The body portion 102 can have a lateral or widthwise dimension of about 18-30 inches or about 22-26 inches. In some configurations, multiple sizes of the leg wrap 100 can be provided to suit a range of users. A first size of the body portion 102 (e.g., S/M) can have a length dimension of about 22-26 inches (e.g., 24 inches) and a second size (e.g., L/XL) can have a length dimension of about 26-28 inches (e.g., 27 inches). A first size of the body portion 102 (e.g., S/M) can have a width dimension of about 20-26 inches and a second size (e.g., L/XL) can have a width dimension of about 22-28 inches. In some implementations the width is constant along the length, while in other implementations the width tapers in a direction from the upper end toward the lower end. In some implementations, an upper portion of the body portion 102 can have a greater width dimension that a lower portion of the body portion 102. For example, in a first size (e.g., S/M) the upper end can have a width dimension of about 26 inches and the bottom end can have a width dimension of about 20 inches. In a second size, (e.g., L/XL) the upper end can have a width dimension of about 28 inches and the bottom end can have a width dimension of about 22 inches.

The leg wrap 100 preferably includes a retention arrangement configured to selectively secure the body portion 102 in a wrapped configuration around the leg of a user. In the illustrated arrangement, the leg wrap 100 includes a plurality of retention elements 114 configured to retain the leg wrap 100 in the wrapped configuration or wrapped orientation on the user's leg. In some implementations, one or more, or each, of the retention elements 114 is a band or strap. The band or strap 114 can be an elastic strap. With such an arrangement, over-tightening of the retention band or strap 114 can be avoided or limited. However, in other implementations, non-elastic straps or other retention devices or arrangements can be used. Each band or strap 114 can have a width of about 1-3 inches or about 1.5-2 inches.

Each of the bands or straps 114 is configured to secure one side of the body portion 102 to the other side of the body portion 102. In the illustrated arrangement, each of the bands or straps 114 can have a first portion on a first side of the body portion 102 and a second portion on a second side of the body portion 102. The first portion and the second portion can be coupled to one another. In some implementations, a hook-and-loop fastener is used to couple the first portion and the second portion of the band or strap 114. In other arrangements, the band or strap 114 can be coupled to one side of the body portion 102 and configured to couple to the other side of the body portion 102, such as with a snap-buckle.

In some implementations, the interior space between the outer layer 104 and the inner layer 106 of the body portion 102 is divided into a plurality of discrete spaces, chambers, or pockets 110 each configured to receive a cold pack 112. The leg wrap 100 can include a reinforcement strip 116 that extends in a lengthwise direction along the body portion 102. The attachment of the reinforcement strip 116 can couple the outer layer 104 and inner layer 106 to segment the interior space into two sides. The leg wrap 100 can also include a plurality of segment lines (e.g., stitch lines) 120 that couple the outer layer 104 and the inner layer 106 to create the multiple chambers or pockets 110 on one or both sides. In some implementations, three stitched segment lines 120 are provided to create four pockets 110 on each side for a total of eight pockets 110. In some configurations, such an arrangement locates four pockets (two on each side) on the user's upper leg and four pockets (two on each side) on the user's lower leg. Another particularly preferred embodiment utilizes two stitched segment lines 120 to create three pockets 110 on each side for a total of six pockets. In some implementations, such an arrangement locates two pockets on the upper leg, two pockets on the lower leg and two pockets at the knee (and possibly extending onto one or both of the upper leg and lower leg). However, other suitable number of pockets 110 can be provided using any desired number of reinforcement strips 116 (or other longitudinal segment lines) and lateral segment lines 120.

The leg wrap 100 includes one or more openings 124 that permit access to the interior pockets 110. In some implementations, a longitudinal access opening 124 is provided on each side of the body portion 102. Each access opening 124 permits access to all of the pockets 110 on the same side as the access opening 124. The access openings 124 can be defined by or within either or both of the outer layer 104 and the inner layer 106. In some implementations, the access openings 124 are defined by or within the outer layer 104. The access openings 124 can be selectively opened and closed. In some implementations, each of the access openings 124 includes a zipper that allows for access and closure.

In some implementations, a pair of leg wraps 100 are sold as a kit to permit the user to apply cold therapy to both legs at the same time. Cold packs 112 can be provided with the leg wrap and configured such that each one of the cold packs 112 is received within one of the chambers or pockets 110. The cold packs 112 can be of any suitable arrangement. Preferably, the cold packs 112 are flexible. The cold packs 112 can be filled with water, water with additives or a suitable synthetic material that is capable of reuse. The wrap could also be configured for use on the user's arm or other body portion (e.g., torso).

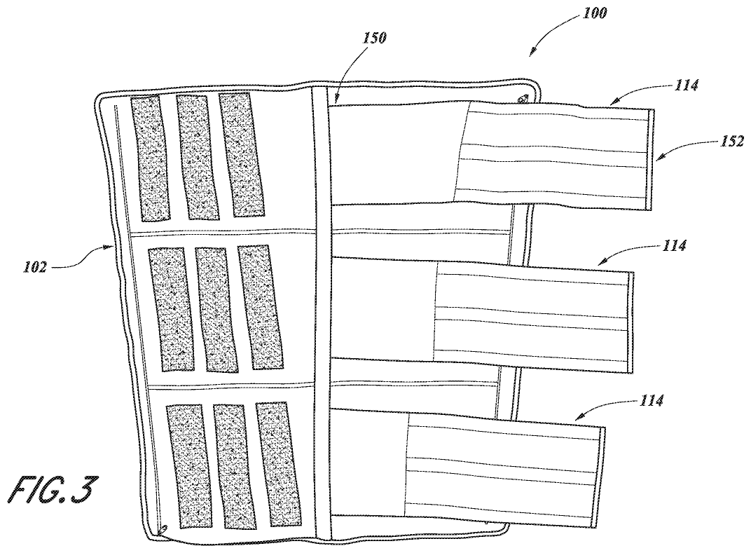

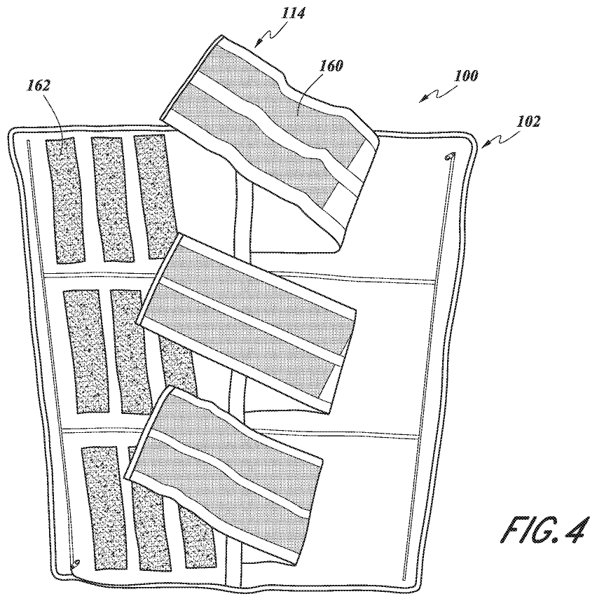

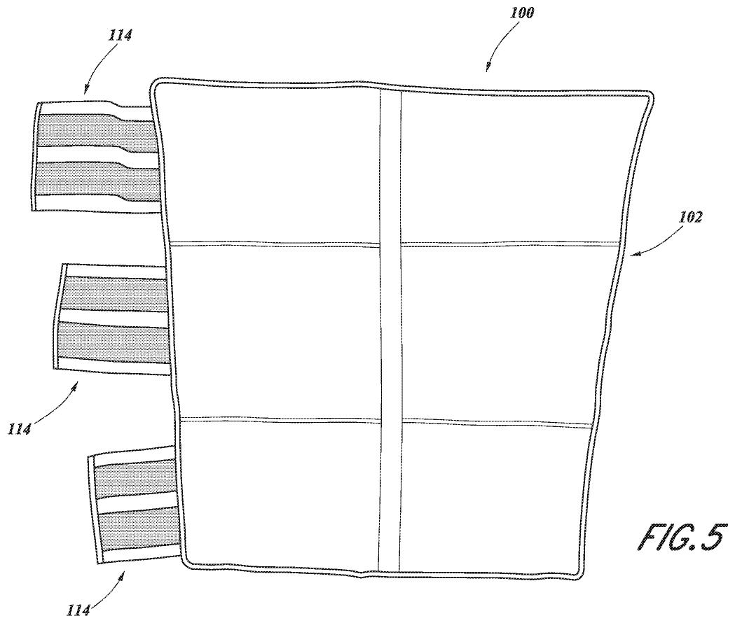

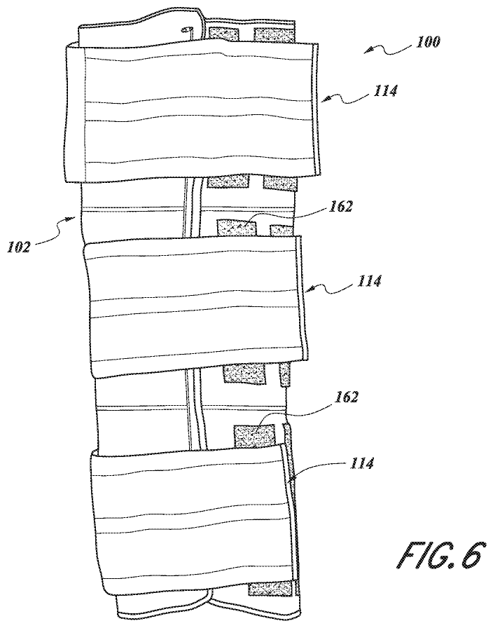

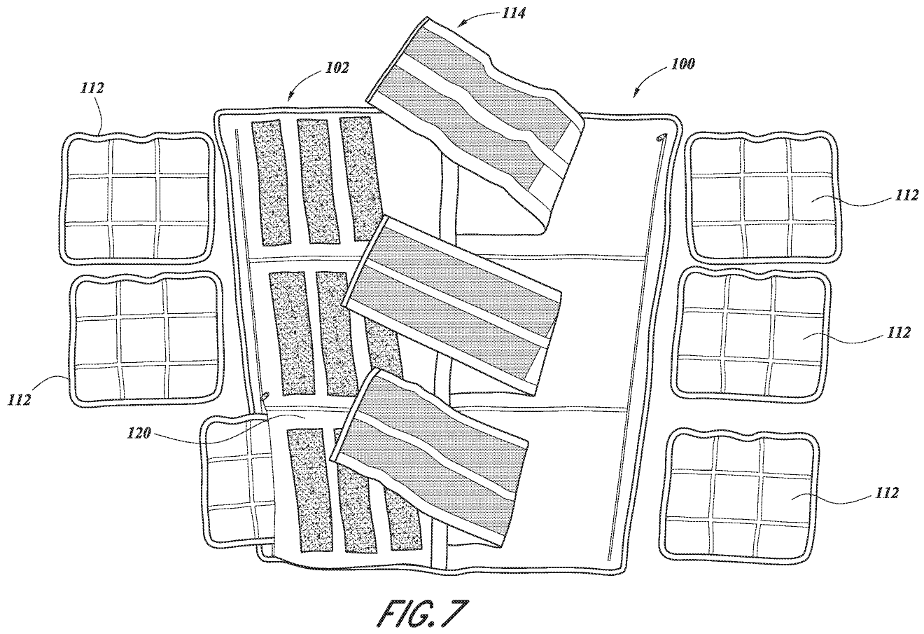

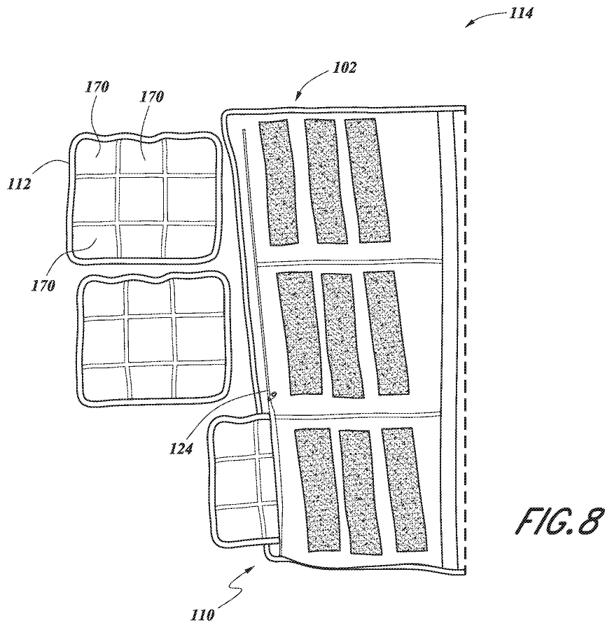

FIGS. 3-8 illustrate another embodiment of the leg wrap 100, which can be similar in many respects to the leg wrap 100 of FIGS. 1 and 2. Accordingly, the leg wrap 100 of FIGS. 3-8 is described in the context of the differences relative to the leg wrap 100 of FIGS. 1 and 2. Any features not described in detail can be the same as or similar to a corresponding feature of the FIGS. 1 and 2, or can be of another suitable arrangement.

The body portion 102 of the leg wrap of FIGS. 3-8 tapers in a direction from an upper end to a lower end. The leg wrap 100 includes at least one retention strap 114. In the illustrated arrangement, the leg wrap 100 includes a plurality of retention straps 114. In particular, the leg wrap 100 includes three retention straps 114 spaced along a length of the leg wrap 100. However, in other arrangements, other numbers of straps 114 may be provided, such as one, two, four or five straps 114, for example.

Each of the straps 114 has an attached end 150 that is fixedly attached to the body portion 102. In the illustrated arrangement, the attached end 150 is permanently secured to the body portion 102. For example, the attached end 150 can be attached to the body portion 102 with a sewn connection. However, in other arrangements the attached end 150 could be removably attached to the body portion 102, such as with a zippered connection, for example.

In the illustrated arrangement, the attached end 150 includes only an endmost portion of the strap 114. That is, only an endmost portion of the strap 114 is attached to the body portion 102. However, in other arrangements, a greater length of the strap 114 can be attached to the body portion 102 so long as the strap 114 is still capable of securing the leg wrap 100 in a wrapped orientation.

Each of the straps 114 defines a free end 152 opposite the attached end 150. In the illustrated arrangement, a substantial entirety of the strap 114, except for the attached end 150, is free relative to the body portion 102. Accordingly, substantial portion or a substantial entirety of the strap 114 is free to move independently of the body portion 102. Such an arrangement permits the orientation of the strap 114 relative to the body portion 102 to be adjusted at least to a small degree, which may enhance the ability of the leg wrap 100 to fit a particular user.

In the illustrated arrangement, a lengthwise direction of the straps 114 are oriented in a widthwise direction of the body portion 102. However, in other arrangements, the straps 114 could be oriented at an oblique angle relative to the body portion 102.

In the illustrated arrangement, the attached end 150 is located at or near a centerline of the body portion 102 in a width direction. The straps 114 extend across one-half or approximately one-half of the body portion 102. Preferably, the straps 114 have a length that is greater than one-half of the width of the body portion 102 such that a portion of each of the straps 114 overhangs the body portion 102 when both are laid flat. In some configurations, a length of the strap 114 may be equal to or greater than about three-quarters of the width of the body portion 102 at the location of the particular strap 114. In some configurations, the length of the straps 114 is less than a width of the body portion 102 at the location of the particular strap 114.

In some configurations, the straps 114 can be constructed from a stretchable or elastic material. In some such configurations, the straps 114 are stretchable at least in the length direction. The straps 114 can also be stretchable in the width direction. However, the degree of stretch may differ between the length direction and the width direction. In some configurations, the straps 114 are constructed in whole or in part by a fabric and foam composite, such as a neoprene or similar material.

The free ends 152 or free end portions of the straps 114 are connectable to the body portion 102 such that the body portion 102 can be secured in a wrapped orientation around a user's leg. The straps 114 and the body portion 102 can each include one half of a hook and loop fastener. For example, the inner surface of the straps 114 include a hook portion 160 of a hook and loop fastener. The outer surface of the body portion 102 can include a loop portion 162 of a hook and loop fastener. The loop portion 162 is located on the opposite side of the body portion 102 from the straps 114. Alternatively, the outer surface of the body portion 102 can be constructed in whole or in part from a loop material that is suitable as one half of the hook and loop fastener.

In the illustrated arrangement, the hook portion 160 on the straps 114 is in the form of one or more strips of hook material, which have a length that extends in a lengthwise direction of the strap 114. In the illustrated arrangement, each strap 114 includes two strips of hook material 160 oriented side by side in the width direction. A combined width of the strips 160 on a particular strap 114 can be at least one-half of the width of the strap 114. A length of the strips of hook material 160 can be at least one-half of the length of the strap 114. A length of the strips of hook material 160 can be about two-thirds of the length of the strap 114.

The loop portion 162 on the body portion 102 is in the form of one or more strips of loop material, which have a length that extends in a lengthwise direction of the body portion 102. Accordingly, the lengthwise direction of the strips of loop material 162 is oriented perpendicular or substantially perpendicular to the lengthwise direction of the strips of hook material 160. In the illustrated arrangement, three strips of loop material 162 are provided and are oriented side by side in a width direction of the body portion 102. A length of the strips of loop material 162 is equal to or greater than a width of the corresponding strap 114. In some configurations, the length of the strips of loop material 162 being greater than a width of the corresponding strap 114 allows for adjustment of an angular orientation of the straps 114 relative to the body portion 102. Such an arrangement allows for some amount of customization of the retention force by the user.

The leg wrap 100 of FIGS. 3-8 includes three interior spaces, chambers or pockets 110 on each side of the body portion 102 for a total of six pockets 110 in the leg wrap 100. The pockets 110 are separate from one another. The pockets 110 can be separated by any suitable arrangement, such as a sewn connection or stitched segment line 120 between the inner layer 106 and the outer layer 104.

Each side of the body portion 102 can include a zippered access opening 124 to the three pockets 110 of the respective side. However, in other arrangements, each pocket 110 has a corresponding, dedicated zippered access opening 124. Moreover, other suitable closures other than or in addition to a zipper can be used.

The cold packs 112 can each include multiple chambers 170 of water, water with additives, synthetic material or other suitable material to provide cold therapy. The chambers 170 can be completely separated or can be connected to one another. The provision of multiple chambers 170 in each cold pack 112 can allow the cold packs 112 to better conform to the user's anatomy. Each of the cold packs 112 can be the same size, even if the size of the pockets 110 may vary, for the sake of manufacturing convenience.

In use, the body portion 102 can be wrapped around a user's leg. If the body portion 102 overlaps itself, the side with the strips of loop material 162 can be kept to the outside of the opposite side so that the strips of loop material 162 remain exposed. Each of the straps 114 can be stretched to a desired length in order to provide a comfortable and secure fit and attached to the strips of loop material 162 to secure the free end 152 to the body portion 102.

### CONCLUSION

It should be emphasized that many variations and modifications may be made to the herein-described embodiments, the elements of which are to be understood as being among other acceptable examples. All such modifications and variations are intended to be included herein within the scope of this disclosure and protected by the following claims. Moreover, any of the steps described herein can be performed simultaneously or in an order different from the steps as ordered herein. Moreover, as should be apparent, the features and attributes of the specific embodiments disclosed herein may be combined in different ways to form additional embodiments, all of which fall within the scope of the present disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Moreover, the following terminology may have been used herein. The singular forms “a,” “an,” and “the” include plural referents unless the context clearly dictates otherwise. Thus, for example, reference to an item includes reference to one or more items. The term “ones” refers to one, two, or more, and generally applies to the selection of some or all of a quantity. The term “plurality” refers to two or more of an item. The term “about” or “approximately” means that quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. The term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “about 1 to 5” should be interpreted to include not only the explicitly recited values of about 1 to about 5, but should also be interpreted to also include individual values and sub-ranges within the indicated range. Thus, included in this numerical range are individual values such as 2, 3 and 4 and sub-ranges such as “about 1 to about 3,” “about 2 to about 4” and “about 3 to about 5,” “1 to 3,” “2 to 4,” “3 to 5,” etc. This same principle applies to ranges reciting only one numerical value (e.g., “greater than about 1”) and should apply regardless of the breadth of the range or the characteristics being described. A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

What is claimed is:

1. A leg wrap, comprising:
an elongate body portion having an outer layer and an inner layer that define one or more interior spaces configured to receive one or more cold packs, wherein the body portion is sized to cover at least a substantial entirety of a length of a user's leg and, when wrapped around the user's leg, to cover at least a substantial entirety of a circumference of the user's leg; and
a plurality of retention elements configured to retain the leg wrap in a wrapped orientation on the user's leg.

2. The leg wrap of claim 1, wherein the body portion is divided into a plurality of discrete pockets, each configured to receive one of the cold packs.

3. The leg wrap of claim 1, further comprising a reinforcement strip that extends in a lengthwise direction along the body portion.

4. The leg wrap of claim 3, wherein the attachment of the reinforcement strip couples the outer layer and inner layer to segment the interior space into two sides.

5. The leg wrap of claim 4, further comprising a plurality of segment lines that couple the outer layer and the inner layer to create multiple pockets on one or both sides created by the reinforcement strip.

6. The leg wrap of claim 5, wherein two or three stitched segment lines are provided to create three or four chambers on each side for a total of six or eight chambers.

7. The leg wrap of claim 5, wherein a longitudinal access opening is provided on each side of the body portion to permit access to all of the pockets on the same side of the access opening.

8. The leg wrap of claim 7, wherein the access openings are defined by or within the outer layer.

9. The leg wrap of claim 7, wherein each of the access openings comprises a zipper that allows for access and closure.

10. The leg wrap of claim 1, wherein the retention elements are bands or straps.

11. The leg wrap of claim 10, wherein each of the bands or straps is an elastic band or strap.

12. The leg wrap of claim 11, wherein each of the bands or straps can have a first portion on a first side of the body portion and a second portion on a second side of the body portion and the first portion and the second portion can be coupled to one another.

13. The leg wrap of claim 12, wherein a hook-and-loop fastener is provided to couple the first portion and the second portion of the band or strap.

14. The leg wrap of claim 11, wherein each of the bands or straps extends along one side of the body portion and connects to the other side of the body portion.

15. The leg wrap of claim 14, wherein an attached end of each of the bands or straps is attached at or near a center of the body portion and wherein a remainder of each of the bands or straps is free of the body portion.

16. The leg wrap of either one of claim 15, wherein a length of each of the bands or straps is greater than one-half of a width of the body portion such that each of the bands or straps overhangs the body portion when the leg wrap is laid flat.

17. The leg wrap of claim 14, wherein each of the bands or straps includes one or more fastener strips oriented in a lengthwise direction of the band or strap, the fastener strips configured to attach to the body portion.

18. The leg wrap of claim 17, further comprising one or more fastener strips oriented in a lengthwise direction of the body portion such that the fastener strips of the bands or straps are perpendicular or substantially perpendicular to the fastener strips of the body portion.

19. The leg wrap of claim 18, wherein the fastener strips of the bands or straps are hook material and the fastener strips of the body portion are loop material of a hook and loop fastener.

20. A kit, comprising a pair of the leg wraps of claim 1.
